(19) United States
(12) Patent Application Publication
(10) Pub. No.: US 2007/0270764 A1
(43) Pub. Date: Nov. 22, 2007

(54) Closed Sterile Drainage Device

(76) Inventor: Angus Buchan Gordon, London (GB)

Correspondence Address: MERCHANT & GOULD PC, P.O. BOX 2903, MINNEAPOLIS, MN, 55402-0903, US

(21) Appl. No.: 10/577,687

(22) PCT Filed: Nov. 1, 2004
(86) PCT No.: PCT/GB04/04606

§ 371 Date: Mar. 22, 2007

(30) Foreign Application Priority Data
Oct. 31, 2003 (GB) 0325535.3

(51) Int. Cl. A61M 1/00

(52) U.S. Cl. 604/236; 604/30; 604/326

(57) ABSTRACT

The present invention relates to a closed drainage device (1) comprising: a pre-evacuated chamber (2); a drainage tube (3) connecting the pre-evacuated chamber (2) to a needle (4); and a clamp (5) attached to the connecting tube (3), wherein the clamp (5) is in a first position that maintains the vacuum of the pre-evacuated chamber and has a second position that does not maintain the vacuum of the pre-evacuated chamber.

A seroma is a mass or swelling caused by the localised accumulation of lymphatic fluid. It is common for fluid to accumulate after surgical procedures where extensive areas of the skin are undermined and potential cavities remain, thereby forming a seroma. This is a particular problem in breast reconstruction, mastectomy, following axillary dissection and also after excision of large soft tissue tumours.

Drainage is recommended to reduce patient discomfort, the risk of wound dehiscence, implant extrusion and infection. The drainage of such seromas is commonly performed using a needle and syringe. This can be a time-consuming process; if a large volume of fluid is present in the seroma, it is often necessary to use several syringes or empty a single syringe repeatedly.

Existing devices are generally intended for long term use, for example, U.S. Pat. No. 4,341,212 discloses a drainage system for the continuous removal of drainage fluid over a relatively long post-operative period of time. Such a drainage device needs to be secured to the patient, and is not suitable for the removal of serous fluid over a short period of time.

The drainage system of U.S. patent application Ser. No. 6024731 discloses a device that has a bellow-type arrangement formed integrally in the cap, to provide the suction force necessary to remove fluid from the body cavity. Such suction means, i.e., the bellow-type arrangement, render the device bulky and cumbersome to use.

U.S. patent application Ser. No. 5073172 discloses a drainage device wherein the suction means can be controlled to ensure that it remains constant. The disadvantage of this system is that, although the vacuum chamber is pre-evacuated, the means to ensure the control of this pressure are in the form of a bellows-type arrangement that renders the device bulky. Another disadvantage of this device is that its use requires assembly of three parts, preventing quick and efficient drainage of a seroma.

It is common that once such devices are removed, fluid may still accumulate in such body cavities at a slower rate. The existing devices are not suitable for the quick removal of such fluid, once the device has already been removed.

It is an object of the present invention to provide a drainage device that is quick and easy to use, a less bulky alternative to previous devices, so that it can be stored more efficiently and is less costly.

In particular it is a simple method of evacuating fluid, that has accumulated after the removal of the drainage devices, that have to be inserted at the time of surgery.

The present invention relates to a closed drainage device comprising a pre-evacuated chamber; a drainage tube connecting the pre-evacuated chamber to a needle and a clamp attached to the connecting drainage tube, wherein the clamp is in a first position that maintains the vacuum of the pre-evacuated chamber and the clamp has a second position that does not maintain the vacuum of the pre-evacuated chamber.

The present invention relates to a closed drainage device comprising a pre-evacuated chamber which is not located within a housing; a drainage tube connecting the pre-evacuated chamber to a needle and a clamp attached to the connecting drainage tube, wherein the clamp is in a first position that maintains the vacuum of the pre-evacuated chamber and the clamp has a second position that does not maintain the vacuum of the pre-evacuated chamber.

Advantageously, the clamp is a sliding clamp.

Preferably, the pre-evacuated chamber is sterile.

Advantageously, the pre-evacuated chamber has a pressure of up to 500 mm Hg.

In preferred embodiments, the pre-evacuated chamber has a volume of 25 cc, 50 cc, 100 cc, 300 cc, 600 cc or 1200 cc.

Advantageously, at least a portion of the pre-evacuated chamber is cylindrical.

Preferably, at least a portion of the pre-evacuated chamber is coiled and/or folded to reduce the overall size of the drainage device.

Advantageously, at least a portion of the pre-evacuated chamber is coiled and/or folded into the form of a “Catherine wheel”.

In an advantageous embodiment, at least one wall of the pre-evacuated chamber is corrugated.

Preferably, the wall of the pre-evacuated chamber is made of plastic.

Preferably, the pre-evacuated chamber has a vacuum port. In an advantageous embodiment, the vacuum port is a plug.

Advantageously, the pre-evacuated chamber has an access port. In a preferred embodiment, the port is a tap.

Preferably, the needle is a gauge needle.

Preferably, the gauge needle is a 19 gauge.

Advantageously, the needle is a venflon needle.

Preferably, the needle is a butterfly needle.

Advantageously, the needle has a Luer-Lock fitting.

Preferably, the drainage tube is connected to the needle by a Luer-Lock.

Advantageously, the drainage tube is connected to the pre-evacuated chamber by a Luer-Lock.

Preferably, the drainage tube is connected to the pre-evacuated chamber at manufacture.

In order that the present invention may be more readily understood, embodiments thereof will now be described, by way of example, with reference to the accompanying drawings in which:

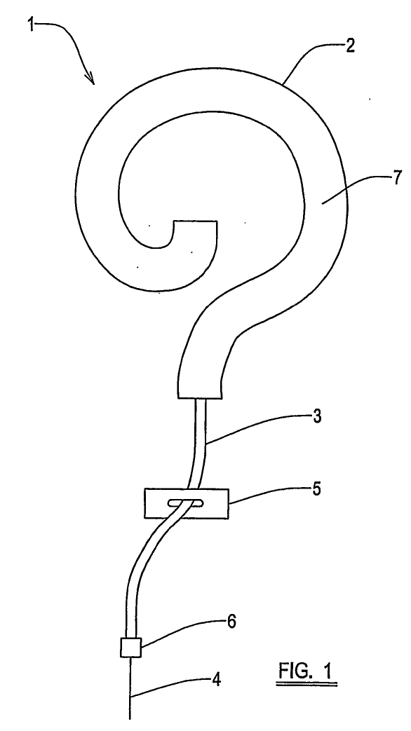

FIG. 1 shows a first embodiment of a closed drainage device in accordance with the present invention; and

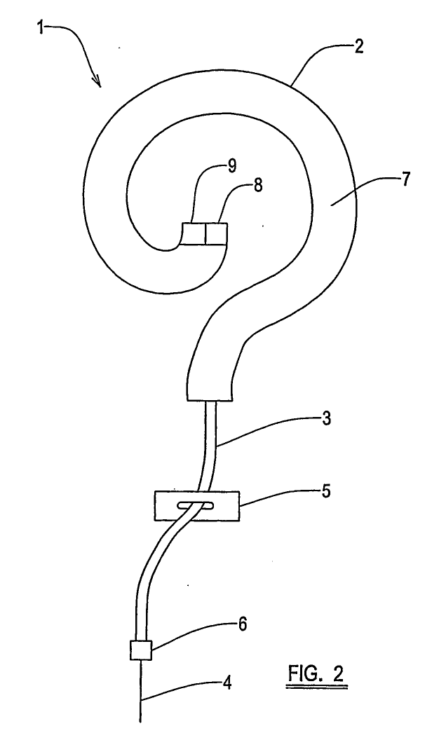

FIG. 2 shows a second embodiment of a closed drainage device in accordance with the present invention.

The present invention relates to a closed drainage device for the removal of fluid from body cavities, in particular, breast cysts, post-operative seromas, pleural effusions and ascites. Referring now to FIG. 1, the closed drainage device (1) comprises a pre-evacuated chamber (2), a drainage tube (3) connecting the pre-evacuated chamber to a needle (4) and a clamp (5), in a closed position, for maintaining the vacuum of the pre-evacuated chamber (2).

In use, to remove fluid from a body cavity, the needle (4) is inserted into the body cavity, in an area of skin that has been adequately prepared. The clamp (5) is moved from the closed position to an open position, thereby causing the fluid to be aspirated from the body cavity, through the needle (4) and drainage tube (3), into the chamber (2). The clamp (5) is moved from the open position to the closed position. The needle (4) is then removed from the body cavity. The entire closed drainage device (1) can be disposed of in tact.

The needle (4), for example, a gauge, venflon or butterfly needle, is connected to the drainage tube (3) by a Luer fitting (6). If the needle is a gauge needle; preferably a 19 gauge is used.

The clamp (5) is any appropriate clamp, for example a sliding clamp or ball type clamp, that can be used in conjunction with the drainage tube (3). The clamp (5) has a closed position, in which it constricts the drainage tube (3) preventing the flow of fluid. The clamp (5) also has an open position, wherein the clamp (5) does not constrict the drainage tube (3) in such a way that prevents the flow of fluid through the drainage tube (3).

The drainage tube (3), preferably an IV type less than 5 mm in diameter, is connected to the chamber (2) and the needle (4) by a standard connector or a Luer-Lock connector. Advantageously, the drainage tube is fixed, at manufacture, to the pre-evacuated chamber.

The chamber (2) can be evacuated by any suitable standard means under sterile conditions. The pressure of the evacuated chamber will be between up to 500 mm Hg. In use, once the clamp (5) is in its open position, the pre-evacuated chamber (2) provides a vacuum assisted suction force.

It is intended that the chamber (2) can be a number of different volumes, for example, 25 cc, 50 cc, 100 cc, 300 cc, 600 cc and 1200 cc, to accommodate different volumes of fluid drainage. The volume of the vacuum chamber (2), of the closed drainage device (1), will depend upon the size of the body cavity or seroma intended to be drained. For example, the 25 cc and 50 cc vacuum chambers (2) could be useful for use in the drainage of a breast cyst, the 100 cc to 600 cc chambers (2) would be suitable for the drainage of seromas of the axilla and back. Renal and/or ovarian cysts could be drained with the appropriate size. The 600 cc and 1200 cc chambers (2) would also be useful for pleural effusions and ascites. It is envisaged that the pressure of the evacuated chamber (2) will vary depending upon the volume of the chamber (2).

Preferably, at least a portion of the vacuum chamber (2) will be cylindrical in shape, to allow the easy flow of fluid into the chamber (2). A cylindrical chamber (2) would enable the volume of fluid to be easily measured. However, it is envisaged that any shape of chamber (2) could be used. Advantageously, at least a portion of the chamber (2) will be coiled and/or folded to reduce the overall size of the device (1). Advantageously, the chamber (2) will be coiled and/or folded into the form of a “Catherine wheel”. In this compact form, more than one closed drainage device (1) could easily be stacked together.

It is also intended that the different size of chambers (2) will have different diameters, i.e. the smaller sized chambers (2), for example, 25 cc, 50 cc and 10 cc will have a smaller diameter than the larger sized chambers(2). Again, this will reduce the overall size of the device (1), making it more compact for storage.

The wall of the chamber (7) will be of sufficient strength to maintain the vacuum of the pre-evacuated chamber. Preferably, the wall of the chamber is made of plastic. A plastic walled chamber (2) will be cheaper to manufacture than a syringe of similar size. Advantageously, at least a portion of the wall of the chamber (7) will be corrugated to provide reinforcement.

With reference to FIG. 2, in a further embodiment of the invention, the chamber (2) has a vacuum port (8), for example a plug, that will provide a point for the evacuation of the chamber, by the standard means discussed above.

The chamber (2) will have a access port (9), for example a tap, to enable the retrieval of a sample of fluid from the chamber (2). Tests may be carried out on such a sample, for example, cytological or microbiological tests. Advantageously, a label will be attached to the external wall of the chamber (2) for the patient's details.

It is intended that the closed drainage device of the present invention will be used for the drainage of fluid from body cavities, in particular, fluid that has accumulated after a surgical procedure. It is intended that the closed drainage device is also applicable to breast cysts, kidney cysts, ovarian cysts, breast / axilla seroma, pleural effusions, ascites and also at other appropriate sites.

It is also envisaged that the closed drainage device will be used, in conjunction with long term drainage devices. In particular, in the event when a long term drainage device has been secured to the patient for long term drainage of fluid and, once such a drainage device has been removed from the patient, a smaller amount of fluid continues to accumulate in the body cavity. It is envisaged that the drainage device of the present invention will be used to drain such fluid.

In the present specification “comprises” means “includes or consists of” and “comprising” means “including or consisting of”.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A closed drainage device comprising:
a pre-evacuated chamber;
a drainage tube connecting the pre-evacuated chamber to a needle; and
a clamp attached to the connecting tube, wherein the clamp is in a first position that maintains the vacuum of the pre-evacuated chamber and has a second position that does not maintain the vacuum of the pre-evacuated chamber.

2. A closed drainage device according to claim 1, wherein the clamp is a sliding clamp.

3. A closed drainage device according to claim 1, wherein the pre-evacuated chamber is sterile.

4. A closed drainage device according to claim 1, wherein the pre-evacuated chamber has a pressure of up to 500 mm Hg.

5. A closed drainage device according to claim 1, wherein the pre-evacuated chamber has a volume of 25 cc, 50 cc, 100 cc, 300 cc, 600 cc or 1200 cc.

6. A closed drainage device according to claim 1, wherein at least a portion of the pre-evacuated chamber is cylindrical.

7. A closed drainage device according to claim 1, wherein at least a portion of the pre-evacuated chamber is coiled and/or folded.

8. A closed drainage device according to claim 7, wherein at least a portion of the pre-evacuated chamber is coiledand/or folded into the form of a “Catherine wheel”.

9. A closed drainage device according to claim 1, wherein the at least one wall of the pre-evacuated chamber is corrugated.

10. A closed drainage device according to claim 1, wherein the at least one wall of the pre-evacuated chamber is made of plastic.

11. A closed drainage device according to claim 1, wherein the pre-evacuated chamber has a vacuum port.

12. A closed drainage device according to claim 1, wherein the vacuum port is a plug.

13. A closed drainage device according to claim 1, wherein the pre-evacuated chamber has an access port.

14. A closed drainage device according to claim 13, wherein the access port is a tap.

15. A closed drainage device according to claim 1, wherein the needle is a gauge needle.

16. A closed drainage device according to claim 15, wherein the gauge needle is a 19 gauge.

17. A closed drainage device according to claim 1, wherein the needle is a venflon needle.

18. A closed drainage device according to claim 1, wherein the needle is a butterfly needle.

19. A closed drainage device according to claim 1, wherein the needle has a Luer Lock fitting.

20. A closed drainage device according to claim 1, wherein the drainage tube is connected to the needle by a Luer-Lock.

21. A closed drainage device according to claim 1, wherein the drainage tube is connected to the pre-evacuated chamber by a Luer-Lock.

22. A closed drainage device according to claim 1 wherein the drainage tube is connected to the pre-evacuated chamber at manufacture.

23. (canceled)
